(19) United States
(12) Patent Application Publication
(10) Pub. No.: US 2008/0234274 A1
(43) Pub. Date: Sep. 25, 2008

(54) Novel Compounds

(75) Inventors: Andrew Bailey, Leicestershire (GB); Garry Pairaudeau, Leicestershire (GB); Anil Patel, Leicestershire (GB); Stephen Thom, Leicestershire (GB)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/132,080

(22) Filed: Jun. 3, 2008

Related U.S. Application Data
(63) Continuation of application No. 10/518,818, filed on Dec. 20, 2004, now abandoned.

(30) Foreign Application Priority Data
Jun. 24, 2002 (SE) 0201977-6

(51) Int. Cl. A61K 31/5375; C07D 241/04; C07D 265/30; C07D 217/24; A61P 25/00; A61K 31/47; A61K 31/495

(52) U.S. Cl. 514/237.5; 544/387; 544/162; 546/146; 514/309; 514/255.01

(57) ABSTRACT

The present invention relates to compounds and compositions for treating diseases associated with cysteine protease activity. The compounds are reversible inhibitors of cysteine proteases S, K, F, L and B. Of particular interest are diseases associated with Cathepsin S. In addition this invention also discloses processes for the preparation of such inhibitors.

The present invention relates to compounds and compositions for treating diseases associated with cysteine protease activity. The compounds are reversible inhibitors of cysteine proteases S, K, F, L and B. Of particular interest are diseases associated with Cathepsin S. In addition this invention also discloses processes for the preparation of such inhibitors.

### BACKGROUND OF THE INVENTION

Cathepsin S is a member of the papain superfamily of cysteine proteases which also encompasses Cathepsins B, H, L, O and K. Cathepsin S plays a key role in the processing of invariant chain in MHC class II complexes allowing the complex to associate with antigenic peptides. MHC class II complexes are then transported to the surface of the cell for presentation to effector cells such as T cells. The process of antigen presentation is a fundamental step in initiation of the immune response. In this respect inhibitors of cathepsin S could be useful agents in the treatment of inflammation and immune disorders such as, but not limited to, asthma, rheumatoid arthritis, multiple sclerosis and Crohn's disease. Cathepsin S has also been implicated in a variety of other diseases involving extracellular proteolysis such as the development of emphysema in COPD through degradation of elastin and in Alzheimers disease.

Other Cathepsins notably K and L have been shown to degrade bone collagen and other bone matrix proteins. Inhibitors of these cysteine proteases would be expected to be useful in the treatment of diseases involving bone resorption such as osteoporosis.

The present invention therefore provides use of a compound of formula (I)

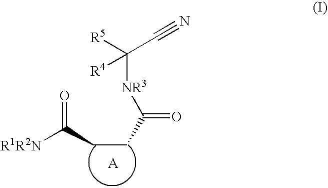

in which:
A is a 6-membered ring optionally containing a double bond and optionally containing an oxygen atom or NR group in the ring;
R is hydrogen or C₁₋₆ alkyl;
R¹ and R² are independently, C₁₋₆ alkyl or C₃₋₆ cycloalkyl both of which can optionally contain one or more O, S or NR³ groups, or R¹ and R² together with the nitrogen atom to which they are attached form a 3,4-dihydroisoquinoline ring or a 5- or 6-membered saturated ring optionally containing a further O, S or N atom and optionally substituted by a group —(CH₂)ₚ—R⁶ where p is 0 to 3 and R⁶ is C₁₋₆ alkyl, CONR⁷R⁵ where R⁷ and R⁵ are independently hydrogen, C₁₋₆ alkyl which can optionally contain one or more O, S or NR³ groups, or together with the nitrogen atom to which they are attached form a 5- or 6-membered saturated ring optionally containing a further O, S or NR³ group; or R⁶ is a 4 to 7-membered saturated ring optionally containing one or more O, S or N atoms, or an aryl or heteroaryl group containing one to four heteroatoms selected from O, S or N, the saturated ring, aryl and heteroaryl groups all being optionally substituted by halogen, amino, hydroxy, cyano, nitro, carboxy, CONR⁷R⁸, SO₂NR⁷R⁸, SO₂R³, trifluoromethyl, NHSO₂R³, NHCOR³, C₁₋₆ alkyl, C₁₋₆ alkoxy, SR³ or NR⁹R¹⁰ where R⁹ and R¹⁰ are independently hydrogen, C₁₋₆ alkyl or together with the nitrogen atom to which they are attached form a 5- or 6-membered saturated ring optionally containing a further O, S or NR³ group;
R³ is hydrogen or C₁₋₆ alkyl;
R⁴ is hydrogen or C₁₋₆ alkyl;
R⁵ is hydrogen, C₁₋₆ alkyl or C₃₋₆ cycloalkyl both of which can optionally contain one or more O, S or NR³ groups or R⁵ is aryl or a 5- or 6-membered heteroaryl group containing one or two heteroatoms selected from O, S or N, the aryl and heteroaryl groups all being optionally substituted by halogen, amino, hydroxy, cyano, nitro, carboxy, CONR⁷R⁸, SO₂NR⁷R⁸, SO₂R³, trifluoromethyl, NHSO₂R³, NHCOR³, C₁₋₆ alkyl, C₁₋₆ alkoxy, SR³ or NR⁹R¹⁰ where R⁹ and R¹⁰ are independently hydrogen, C₁₋₆ alkyl or together with the nitrogen atom to which they are attached form a 5- or 6-membered saturated ring optionally containing a further O, S or NR³ group;
or R⁴ and R⁵ together form a 5- or 6-membered saturated ring optionally containing a further O, S or NR³ group and optionally substituted by, C₁₋₆ alkyl;
and pharmaceutically acceptable salts or solvates thereof, in the manufacture of a medicament for use in the inhibition of Cathepsin S in a warm blooded animal, such as man.

In the context of the present specification, unless otherwise indicated, an alkyl or alkenyl group or an alkyl or alkenyl moiety in a substituent group may be linear or branched. Aryl groups include phenyl and naphthyl. Heteroaryl groups include 5- or 6-membered, 5,6- or 6,6-fused aromatic rings containing one or more heteroatoms selected from N, S, O. Examples include pyridine, pyrimidine, pyrazine, pyridazine thiazole, oxazole, pyrazole, imidazole, furan and thiophene, quinoline, isoquinoline, benzimidazole, benzofuran, benzothiophene, indole.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

Suitably A is a 6-membered ring optionally containing a double bond and optionally containing an oxygen atom or NR group in the ring where R is hydrogen or C₁₋₆ alkyl. A double bond can be present in any suitable position of the ring A. An oxygen atom or NR group can be present in any suitable position of the ring A, in addition to a double bond if desired. Preferably A is a cyclohexane ring.

Preferably R¹ and R² together with the nitrogen atom to which they are attached form an unsubstituted morpholine ring or a piperidine or piperazine ring substituted by a group —(CH₂)ₚ—R⁶ where p and R⁶ are as defined above. Preferably p is 0 and R⁶ is aryl or heteroaryl optionally substituted as defined above.

Preferably R³ is hydrogen.

Preferably R⁴ is hydrogen.

Preferably R⁵ is hydrogen, phenyl optionally substituted by C₁₋₆ alkyl or C₁₋₆ alkoxy

Preferred compounds of the invention include:
(1R,2R)—N-[Cyano(2-methoxyphenyl)methyl]-2-(morpholin-4-ylcarbonyl)cyclohexanecarboxamide,
(1R,2R)—N-[Cyano(2-methoxyphenyl)methyl]-2-{[4-(4-fluorobenzyl)piperazin-1-yl]carbonyl}cyclohexane carboxamide,
(1R,2R)—N-[Cyano(2-methoxyphenyl)methyl]-2-(3,4-dihydroisoquinolin-2(1H)-ylcarbonyl)cyclohexane carboxamide,
(±) Trans-N-(cyanomethyl)-2-{[4-(4-fluorobenzyl)piperazin-1-yl]carbonyl}cyclohexanecarboxamide,
(±) Trans-N-[cyano(2-methoxyphenyl)methyl]-2-[(4-methylpiperazin-1-yl)carbonyl]cyclohexanecarboxamide,
(1R,2R)—N-[Cyano(2-methoxyphenyl)methyl]-2-{[4-(4-fluorophenyl)piperazin-1-yl]carbonyl}cyclohexane carboxamide,
(1R,2R)—N-(4-Cyano-1-methylpiperidin-4-yl)-2-{[4-(4-fluorophenyl)piperazin-1-yl]carbonyl}cyclohexane carboxamide,
(1R,2R)—N-(4-Cyanotetrahydro-2H-pyran-4-yl)-2-{[4-(4-fluorophenyl)piperazin-1-yl]carbonyl}cyclohexane carboxamide,
(1R,2R)—N-[(1S)-1-cyano-3-methoxypropyl]-2-{[4-(4-fluorophenyl)piperazin-1-yl]carbonyl}cyclohexanecarboxamide,
   and pharmaceutically acceptable salts thereof.

The present invention further provides a process for the preparation of a compound of formula (I) which comprises reaction of a compound of general formula (II) with a dehydrating agent (e.g. phosphorous oxychloride)

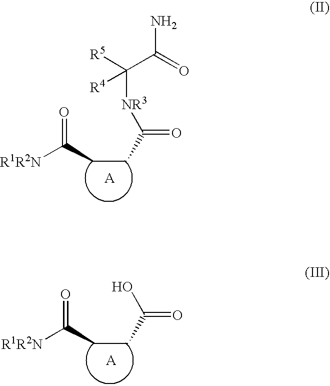

Compounds of formula (II) may be prepared from compounds of formula (III) by activation of the acid with an appropriate coupling agent or formation of acid chloride followed by reaction with an amine HNR³(CR⁴R⁵)CONH₂ where R³, R⁴ and R⁵ are defined in formula (I)

reaction of a compound of general formula (III) by activation of the acid group with an appropriate coupling agent or formation of acid chloride followed by reaction with an amine HNR³(CR⁴R⁵)CN where R³, R⁴ and R⁵ are defined in formula (I)
reaction of a compound of general formula (IV), where X═CN or CONH₂, by activation of the acid group with an appropriate coupling agent or formation of acid chloride followed by reaction with an amine HNR¹R² where R¹ and R² are defined in formula (I)

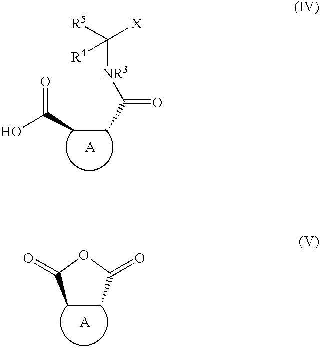

Compounds of general formula (III) and (IV) may be prepared from compound of general formula (V) by reaction with an amine of general formula HNR³(CR⁴R⁵)CN, HNR³(CR⁴R⁵)CONH₂ where R³, R⁴ and R⁵ are defined in formula (I) or HNR¹R² where R¹ and R² are defined in formula (I).

Compounds of general formula (III) and (IV) may also be prepared from a compound of general formula (VI) by activation of the acid group with an appropriate coupling agent or formation of acid chloride followed by reaction with an amine of general formula HNR³(CR⁴R⁵)CN, HNR³(CR⁴R⁵)CONH₂ where R³, R⁴ and R⁵ are defined in formula (I) or HNR¹R² where R¹ and R² are defined in formula (I) followed by hydrolysis of the ester.

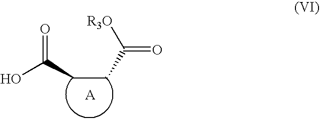

According to a further feature of the invention there is provided a compound of the formula (I), or a pharmaceutically acceptable salt thereof, for use as a therapeutic agent.

According to a further feature of the present invention there is provided a method for producing inhibition of a cysteine protease in a warm blooded animal, such as man, in need of such treatment, which comprises administering to said animal an effective amount of a compound of the present invention, or a pharmaceutically acceptable salt thereof. In particular the compounds of the invention are useful in the treatment of inflammation and immune disorders such as, but not limited to, asthma, rheumatoid arthritis, COPD, multiple sclerosis, Crohn's disease, Alzheimers and pain, such as neuropathic pain. Preferably the compounds of the invention are used to treat pain, especially neuropathic pain.

The invention also provides a compound of the formula (I), or a pharmaceutically acceptable salt thereof, for use as a medicament; and the use of a compound of the formula (I) of the present invention, or a pharmaceutically acceptable salt thereof, in the manufacture of a medicament for use in the inhibition of a cysteine protease in a warm blooded animal, such as man.

In particular the invention provides the use of a compound of the formula (I) of the present invention, or a pharmaceutically acceptable salt thereof, in the manufacture of a medicament for use in the inhibition of Cathepsin S in a warm blooded animal, such as man. In order to use a compound of the formula (I) or a pharmaceutically acceptable salt thereof for the therapeutic treatment of mammals including humans, in particular in the inhibition of a cysteine protease, it is normally formulated in accordance with standard pharmaceutical practice as a pharmaceutical composition.

Therefore in another aspect the present invention provides a pharmaceutical composition which comprises a compound of the formula (I) or a pharmaceutically acceptable salt thereof and a pharmaceutically acceptable diluent or carrier.

The pharmaceutical compositions of this invention may be administered in standard manner for the disease condition that it is desired to treat, for example by oral, rectal or parenteral administration. For these purposes the compounds of this invention may be formulated by means known in the art into the form of, for example, tablets, capsules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 100 mg and 1 g of the compound of this invention.

In another aspect a pharmaceutical composition of the invention is one suitable for intravenous, subcutaneous or intramuscular injection.

Each patient may receive, for example, an intravenous, subcutaneous or intramuscular dose of 1 mgkg⁻¹ to 100 mgkg⁻¹ of the compound, preferably in the range of 5 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the compound of formula (I), or a pharmaceutically-acceptable salt thereof (hereafter compound X), for therapeutic or prophylactic use in humans:

(a)

| Tablet I | mg/tablet |
|---|---|
| Compound X. | 100 |
| Lactose Ph.Eur. | 179 |
| Croscarmellose sodium | 12.0 |
| Polyvinylpyrrolidone | 6 |
| Magnesium stearate | 3.0 |

(b)

| Tablet II | mg/tablet |
|---|---|
| Compound X | 50 |
| Lactose Ph.Eur. | 229 |
| Croscarmellose sodium | 12.0 |
| Polyvinylpyrrolidone | 6 |
| Magnesium stearate | 3.0 |

(c)

| Tablet III | mg/tablet |
|---|---|
| Compound X | 1.0 |
| Lactose Ph.Eur. | 92 |
| Croscarmellose sodium | 4.0 |
| Polyvinylpyrrolidone | 2.0 |
| Magnesium stearate | 1.0 |

(d)

| Capsule | mg/capsule |
|---|---|
| Compound X | 10 |
| Lactose Ph.Eur. | 389 |
| Croscarmellose sodium | 100 |
| Magnesium stearate | 1. |

(e)

| Injection I | (50 mg/ml) |
|---|---|
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β cyclodextrin may be used to aid formulation.

### Note

The above formulations may be obtained by conventional procedures well known in the pharmaceutical art. The tablets (a)-(c) may be enteric coated by conventional means, for example to provide a coating of cellulose acetate phthalate.

The following examples illustrate the invention.

### EXAMPLE 1

### (1R,2R)—N-[Cyano(2-methoxyphenyl)methyl]-2-(morpholin-4-ylcarbonyl)cyclohexanecarboxamide

### (i) (1R,2R)-2-({[Cyano(2-methoxyphenyl)methyl]amino}carbonyl)cyclohexanecarboxylic acid

A mixture of (3aR,7aR)-hexahydro-2-benzofuran-1,3-dione (3.64 g), N,N-diisopropylethylamine (7.63 g) and (±) 2-(2-methoxyphenyl)aminoacetonitrile hydrochloride (4.7 g) in tetrahydrofuran (50 ml) was stirred at room temperature for 6 hours. The solvent was removed under reduced pressure and the residue dissolved in water. The cooled (0° C.) aqueous solution was acidified by dropwise addition of dilute aqueous hydrochloric acid and the resultant mixture extracted with ethyl acetate. The organic layer was washed with aqueous brine, dried (MgSO4), and evaporated under reduced pressure. The residue was purified by tritration with diethyl ether (100 ml) followed by ethyl acetate (3×30 ml). Yield 1.5 g

MS: APCI (+ve) 317 (M+1)

### (ii) (1R,2R)—N-[Cyano(2-methoxyphenyl)methyl]-2-(morpholin-4-ylcarbonyl)cyclohexanecarboxamide

A solution of the product from step (i) (0.35 g), morpholine (0.14 g), N,N-diisopropylethylamine (0.36 g) and 1-hydroxybenzotriazole (0.22 g) in tetrahydrofuran (10 ml) was treated with N-(3-dimethylaminopropyl)-N′-ethylcarbodiimide hydrochloride (0.32 g) and stirred for 6 hours at room temperature. The mixture was partitioned between ethyl acetate and aqueous brine, the organics dried (MgSO4) and evaporated under reduced pressure. The residue was purified by chromatography on silica, eluting with a mixture of ethyl acetate (75%) and isohexane (25%). Yield 0.05 g

MS: APCI (+ve) 386 (M+1)

1H NMR: (CDCl₃) δ 7.37-7.29 (2H, m), 6.96-6.92 (3H, m), 6.07 (1H, d), 3.97 (3H, s), 3.53-3.28 (6H, m), 3.16-3.12 (2H, m), 2.77-1.63 (2H, m), 1.95 (1H, d), 1.84-1.58 (4H, m), 1.46-1.26 (3H, m).

### EXAMPLES 2 AND 3

Examples 2 and 3 were prepared according to the general method of example 1 using the appropriate amines

### EXAMPLE 2

### (1R,2R)—N-[Cyano(2-methoxyphenyl)methyl]-2-{[4-(4-fluorobenzyl)piperazin-1-yl]carbonyl}cyclohexane carboxamide

MS: APCI (+ve) 493 (M+1)

1H NMR: (CDCl₃) δ 7.37-7.21 (4H, m), 7.03-6.92 (5H, m), 6.06 (1H, d), 3.96 (3H, s), 3.36-3.31 (5H, m), 3.20-3.10 (1H, m), 2.73-2.66 (2H, m), 2.29-2.25 (2H, m), 2.20-2.12 (1H, m), 1.95-1.60 (6H, m), 1.40-1.20 (3H, m).

### EXAMPLE 3

### (1R,2R)—N-[Cyano(2-methoxyphenyl)methyl]-2-(3,4-dihydroisoquinolin-2(1H)-ylcarbonyl)cyclohexane carboxamide

MS: APCI (+ve) 432 (M+1)

1H NMR: (DMSO-d6) δ 9.06-8.97 (1H, m), 7.42-7.32 (2H, m), 7.23-6.93 (6H, m), 6.03-5.93 (1H, m), 4.73-4.38 (2H, m), 3.81, 3.73 (3H, 2×S), 3.80-3.40 (2H, m), 3.00-2.55 (4H, m), 1.92-1.69 (4H, m), 1.38-1.17 (4H, m).

### EXAMPLE 4

### (±) Trans-N-(cyanomethyl)-2-{[4-(4-fluorobenzyl)piperazin-1-yl]carbonyl}cyclohexanecarboxamide

A mixture of (±)trans-1,2-cyclohexanedicarboxylic anhydride (0.4 g), N,N-diisopropylethylamine (0.34 g) and 1-(4-fluorobenzyl)piperazine (0.5 g) in tetrahydrofuran (15 ml) was stirred at room temperature for 18 hours. At the end of this time the reaction mixture was treated with further N,N-diisopropylethylamine (0.84 g) followed by aminoacetonitrile hydrochloride (0.36 g), 1-hydroxybenzotriazole (0.53 g) and finally N-(3-dimethylaminopropyl)-N′-ethylcarbodiimide hydrochloride (0.75 g). The mixture was stirred for 18 hours and subsequently partitioned between ethyl acetate and aqueous sodium bicarbonate, the organics dried (MgSO4) and evaporated under reduced pressure. The residue was purified by chromatography on silica, eluting with a mixture of triethylamine (0.6%), methanol (2%) and dichloromethane (97.4%). Yield 0.045 g

MS: APCI (+ve) 387 (M+1)

1H NMR: (DMSO-d6) δ 8.46 (1H, t), 7.36-7.31 (2H, m), 7.17-7.11 (2H, m), 4.03 (2H, d), 3.50-3.35 (6H, m), 2.90-2.80 (1H, m), 2.41-2.24 (4H, m), 1.80-1.77 (1H, m), 1.73-1.65 (3H, m), 1.32-1.15 (4H, m).

### EXAMPLE 5

### (±) Trans-N-[cyano(2-methoxyphenyl)methyl]-2-[(4-methylpiperazin-1-yl)carbonyl]cyclohexanecarboxamide

### (i) (±) Trans-2-({[cyano(2-methoxyphenyl)methyl]amino}carbonyl)cyclohexanecarboxylic acid

A mixture of (±) trans-1,2-cyclohexanedicarboxylic anhydride (3.0 g), N,N-diisopropylethylamine (5.03 g) and (±) 2-(2-methoxyphenyl)aminoacetonitrile hydrochloride (3.87 g) in tetrahydrofuran (50 ml) was stirred at room temperature for 18 hours. The solvent was removed under reduced pressure and the residue dissolved in water. The cooled (0° C.) aqueous solution was acidified by dropwise addition of dilute aqueous hydrochloric acid and the resultant mixture extracted with ethyl acetate. The organic layer was washed with aqueous brine, dried (MgSO4), and evaporated under reduced pressure. The residue was purified by tritration with ethyl acetate (2×30 ml). Yield 0.53 g

MS: APCI (+ve) 317 (M+1)

### (ii) (1R,2R)—N-[Cyano(2-methoxyphenyl)methyl]-2-[(4-methylpiperazin-1-yl)carbonyl]cyclohexane carboxamide

A solution of the product from step (i) (0.38 g), 1-methylpiperazine (0.18 g), N,N-diisopropylethylamine (0.23 g) and 1-hydroxybenzotriazole (0.24 g) in 1-methyl-2-pyrrolidinone (10 ml) was treated with N-(3-dimethylaminopropyl)-N′-ethylcarbodiimide hydrochloride (0.35 g) and stirred for 4 hours at room temperature. The mixture was partitioned between ethyl acetate and aqueous sodium bicarbonate, the organics washed with aqueous brine (×3), dried (MgSO4) and evaporated under reduced pressure. The residue was purified by chromatography on silica, eluting with a mixture of triethylamine (0.4%), methanol (4%) and dichloromethane (95.6%) followed by tritration of the resultant product with diethyl ether. Yield 0.035 g

MS: APCI (+ve) 399 (M+1)

1H NMR: (DMSO-d6) δ 8.99 (1H, d), 7.44-7.38 (2H, m), 7.09 (1H, d), 7.00 (1H, t), 6.00 (1H, d), 3.85 (3H, s), 3.44-3.40 (2H, m), 2.90-2.82 (1H, m), 2.70-2.62 (1H, m), 2.24-2.21 (2H, m), 2.11-2.08 (4H, m), 2.03-2.01 (1H, m), 1.87 (1H, d), 1.76-1.66 (3H, m), 1.36-1.17 (4H, m).

### EXAMPLE 6

### (1R,2R)—N-[Cyano(2-methoxyphenyl)methyl]-2-{[4-(4-fluorophenyl)piperazin-1-yl]carbonyl}cyclohexane carboxamide

### (i) Methyl (1R,2R)-2-{[4-(4-fluorophenyl)piperazin-1-yl]carbonyl}cyclohexanecarboxylate

A solution of (1R,2R)-cyclohexane 1,2-dicarboxylic acid mono-methyl ester (1.0 g) in 1-methyl-2-pyrrolidinone (20 ml) was treated with N,N-diisopropylethylamine (1.73 g) followed by 1-(4-fluorophenyl)piperazine (1.45 g) and 1-hydroxybenzotriazole (1.09 g). The resultant mixture was then treated with N-(3-dimethylaminopropyl)-N′-ethylcarbodiimide hydrochloride (1.55 g) and stirred for 18 hours at room temperature. The mixture was partitioned between ethyl acetate and water, the organics washed with water (×3), dried (MgSO4) and evaporated under reduced pressure. The residue was purified by chromatography on silica, eluting with a mixture of ethyl acetate (45%) and isohexane (55%). Yield 1.4 g

MS: APCI (+ve) 349 (M+1)

### (ii) (1R,2R)-2-{[4-(4-Fluorophenyl)piperazin-1-yl]carbonyl}cyclohexanecarboxylic acid

A solution of the product from step (i) (1.1 g) in methanol (40 ml) was treated with a solution of sodium hydroxide (0.25 g) in water (20 ml) and the resultant mixture heated at 50° C. for 24 hours. The solvent was removed under reduced pressure and the residue dissolved in water and washed with diethyl ether, the aqueous layer was acidified by addition of glacial acetic acid and extracted with ethyl acetate (×2). The combined ethyl acetate layers were washed with aqueous brine, dried (MgSO4) and evaporated under reduced pressure. The residue was purified by tritration with diethyl ether. Yield 0.9 g

MS: APCI (+ve) 335 (M+1)

### (iii) (1R,2R)—N-[Cyano(2-methoxyphenyl)methyl]-2-{[4-(4-fluorophenyl)piperazin-1-yl]carbonyl}cyclohexanecarboxamide

A solution of the product from step (iii) (0.35 g) in dichloromethane (10 ml) at 0° C. was treated with (±) 2-(2-methoxyphenyl)aminoacetonitrile hydrochloride (0.25 g) and N,N-diisopropylethylamine (0.54 g) followed by O-(7-Azabenzotriazol-1-yl)-N,N,N′,N′-tetramethyluronium hexafluorophosphate (0.44 g). The mixture was stirred at 0° C. for 1 hour and then at room temperature for 18 hours. The mixture was partitioned between ethyl acetate and water, the organics washed with water, dried (MgSO4) and evaporated under reduced pressure. The residue was purified by chromatography on silica, eluting with a mixture of ethyl acetate (67%) and isohexane (33%). Yield 0.27 g

MS: APCI (+ve) 479 (M+1)

1H NMR: (CDCl₃) δ 7.39-7.23 (2H, m), 7.01-6.77 (7H, m), 6.06-5.97 (1H, m), 3.97-3.95 (4H, m), 3.80-3.36 (4H, m), 3.19-3.15 (1H, m), 3.10-2.47 (5H, m), 1.98-1.75 (3H, m), 1.65-1.26 (4H, m).

### EXAMPLES 7 AND 8

Examples 7 and 8 were prepared according to the general method of example 6 step (iii) using the appropriate amines.

### EXAMPLE 7

### (1R,2R)—N-(4-Cyanotetrahydro-2H-pyran-4-yl)-2-{[4-(4-fluorophenyl)piperazin-1-yl]carbonyl}cyclohexane carboxamide

MS: APCI (+ve) 443 (M+1)

1H NMR: (CDCl₃) δ 7.00-6.94 (2H, m), 6.89-6.85 (2H, m), 6.32 (1H, s), 3.90-3.80 (3H, m), 3.78-3.59 (5H, m), 3.16-3.12 (1H, m), 3.09-3.00 (3H, m), 2.92-2.86 (1H, m), 2.70-2.64 (1H, m), 2.46-2.42 (1H, m), 2.22-2.19 (1H, m), 1.94-1.82 (6H, m), 1.69-1.63 (1H, m), 1.49-1.30 (3H, m).

### EXAMPLE 8

### (1R,2R)—N-(4-Cyano-1-methylpiperidin-4-yl)-2-{[4-(4-fluorophenyl)piperazin-1-yl]carbonyl}cyclohexane carboxamide

MS: APCI (+ve) 456 (M+1)

1H NMR: (CDCl₃) δ 7.00-6.94 (2H, m), 6.90-6.85 (2H, m), 6.20 (1H, s), 3.90-3.85 (1H, m), 3.80-3.75 (1H, m), 3.70-3.60 (2H, m), 3.18-3.13 (1H, m), 3.09-3.04 (3H, m), 2.91-2.85 (1H, m), 2.73-2.61 (3H, m), 2.49-2.35 (3H, m), 2.29 (3H, s), 2.28-2.22 (1H, m), 1.90-1.81 (6H, m), 1.70-1.60 (1H, m), 1.50-1.32 (3H, m).

### EXAMPLE 9

### (1R,2R)—N-[(1S)-1-cyano-3-methoxypropyl]-2-{[4-(4-fluorophenyl)piperazin-1-yl]carbonyl}cyclohexanecarboxamide

### (i) N˜2˜(tert-butoxycarbonyl)-O-methyl-L-homoserinamide

A solution of Boc-O-methyl-L-homoserine (7.75 g) in dichloromethane (100 ml) was treated with carbonyldiimidazole (6.46 g) and the mixture stirred for 1 h at room temperature. At the end of this time concentrated aqueous ammonia (20 ml) was added and stirring continued for a further 40 min. The reaction mixture was washed with water followed by dilute aqueous sodium hydroxide and then with aqueous brine before being dried (MgSO4) and evaporated under reduced pressure. Yield 2.9 g

1H NMR: (DMSO-d6) δ 7.22 (1H, s), 6.94 (1H, s), 6.76 (1H, d), 3.94-3.88 (1H, m), 3.20 (3H, s), 1.87-1.83 (1H, m), 1.69-1.64 (1H, m), 1.38 (9H, s).

### (ii) O-Methyl-L-homoserinamide hydrochloride

A solution of the product from step (i) (2.9 g) in 1,4-dioxane (30 ml) was treated with a 4.0 molar solution of HCl in 1,4-dioxane (15 ml) and the mixture allowed to stand for 18 h at room temperature. The resultant precipitate was filtered off and washed with diethylether. Yield 1.84 g

1H NMR: (DMSO-d6) δ 8.20 (3H, s), 7.94 (1H, s), 7.54 (1H, s), 3.76 (1H, s), 3.46-3.37 (2H, m), 3.24 (3H, s), 2.05-1.90 (2H, m).

### (iii) (1R,2R)—N-[(1S)-1-Carboxamide-3-methoxypropyl]-2-{[4-(4-fluorophenyl)piperazin-1-yl]carbonyl}cyclohexanecarboxamide

A solution of the product from step (ii) (0.36 g) in 1-methyl-2-pyrrolidinone (15 ml) was treated with N,N-diisopropylethylamine (0.93 g) followed by (1R,2R)-2-{[4-(4-fluorophenyl)piperazin-1-yl]carbonyl}cyclohexanecarboxylic acid (prepared as described in Example 6, step (ii)) (0.6 g). The reaction mixture was cooled to 0° C. and treated with O-(7-Azabenzotriazol-1-yl)-N,N,N′,N′-tetramethyluronium hexafluorophosphate (0.75 g).

The mixture was stirred at 0° C. for 1 h and then at room temperature for 18 h. The mixture was partitioned between ethyl acetate and water, the organics washed with water (×3), dried (MgSO4) and evaporated under reduced pressure. The residue was purified by trituration with diethylether. Yield 0.40 g

MS: APCI (+ve) 449 (M+1)

### (iv) (1R,2R)—N-[(1S)-1-Cyano-3-methoxypropyl]-2-{[4-(4-fluorophenyl)piperazin-1-yl]carbonyl}cyclohexanecarboxamide

Oxalyl chloride (0.34 g) was added dropwise to N,N-dimethylformamide (10 ml) at 0° C. and the mixture stirred at this temperature for 5 minutes. Pyridine (0.42 g) was then added and stirring continued for a further 5 minutes. At the end of this time the mixture was treated dropwise with a solution of the product of step (iii) (0.59 g) in N,N-dimethylformamide (5 ml). After stirring for 2 hours at 0° C. the mixture was partitioned between ethyl acetate and water, the organics washed with water, dried (MgSO4) and evaporated under reduced pressure. The residue was purified by chromatography on silica, eluting with a mixture of ethyl acetate (80%) and isohexane (20%). Yield 0.21 g

MS: APCI (+ve) 431 (M+1)

1H NMR: (DMSO-d6) δ 8.57 (1H, d), 7.08-7.03 (2H, m), 6.99-6.94 (2H, m), 4.71 (1H, q), 3.70-3.58 (3H, m), 3.50-3.47 (1H, m), 3.37-3.30 (2H, m), 3.17 (3H, s), 3.08-3.00 (3H, m), 2.96-2.89 (2H, m), 2.57-2.49 (1H, m), 1.98-1.68 (6H, m), 1.38-1.20 (4H, m).

### Measurement of Cathepsin S Activity.

QFRET Technology (Quenched Fluorescent Resonance Energy Transfer) was used to measure the inhibition by test compounds of Cathepsin S-mediated cleavage of the synthetic peptide Z-Val-Val-Arg-AMC. Compounds were screened at five concentrations in duplicate and the pIC₅₀ values reported.

Synthetic substrate, 20 μM [final]Z-Val-Val-Arg-AMC in phosphate buffer were added to a 96 well black Optiplate. The assay plates were pre-read for compound auto fluorescence on SpectraMax Gemini at 355 nM excitation and 460 nM emission. 250 μM [final] rHuman Cathepsin S in phosphate buffer was added and incubated for 2 h at room temperature on the SpectraMax Gemini, taking readings every 20 min at 355 nM excitation and 460 nM emission.

Activity Based template (5PTB-8) used the auto fluorescent corrected data to calculate the percentage inhibition for each compound concentration using the relevent plate controls. This data was used to construct inhibition curves and pIC₅₀ estimated by non-linear regression using a 4 parameter logistic model.

1-12. (canceled)

13. A compound of formula (I): 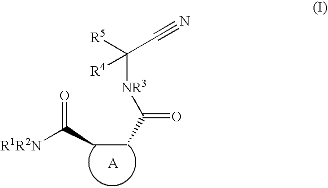
in which:
A is a 6-membered ring optionally containing a double bond and optionally containing an oxygen atom or NR group in the ring;
R is hydrogen or C₁₋₆ alkyl;
R¹ and R² are independently, C₁₋₆ alkyl or C₃₋₆ cycloalkyl both of which can optionally contain one or more O, S or NR³ groups, or R¹ and R² together with the nitrogen atom to which they are attached form a 3,4-dihydroisoquinoline ring or a 5- or 6-membered saturated ring optionally containing a further O, S or N atom and optionally substituted by a group —(CH₂)ₚ—R⁶ where p is 0 to 3 and R⁶ is C₁₋₆ alkyl, CONR⁷R⁸ where R⁷ and R⁸ are independently hydrogen, C₁₋₆ alkyl which can optionally contain one or more O, S or NR³ groups, or together with the nitrogen atom to which they are attached form a 5- or 6-membered saturated ring optionally containing a further O, S or NR³ group;
or R⁶ is a 4 to 7-membered saturated ring optionally containing one or more O, S or N atoms, or an aryl or heteroaryl group containing one to four heteroatoms selected from O, S or N, the saturated ring, aryl and heteroaryl groups all being optionally substituted by halogen, amino, hydroxy, cyano, nitro, carboxy, CONR⁷R⁸, SO₂NR⁷R⁸, SO₂R³, trifluoromethyl, NHSO₂R³, NHCOR³, C₁₋₆ alkyl, C₁₋₆ alkoxy, SR³ or NR⁹R¹⁰ where R⁹ and R¹⁰ are independently hydrogen, C₁₋₆ alkyl or together with the nitrogen atom to which they are attached form a 5- or 6-membered saturated ring optionally containing a further O, S or NR³ group;
R³ is hydrogen or C₁₋₆ alkyl;
R⁴ is hydrogen or C₁₋₆ alkyl;
R⁵ is hydrogen, C₁₋₆ alkyl or C₃₋₆ cycloalkyl both of which can optionally contain one or more O, S or NR³ groups or R⁵ is aryl or a 5- or 6-membered heteroaryl group containing one or two heteroatoms selected from O, S or N, the aryl and heteroaryl groups all being optionally substituted by halogen, amino, hydroxy, cyano, nitro, carboxy, CONR⁷R⁸, SO₂NR⁷R⁸, SO₂R³, trifluoromethyl, NHSO₂R³, NHCOR³, C₁₋₆ alkyl, C₁₋₆ alkoxy, SR³ or NR⁹R¹⁰ where R⁹ and R¹⁰ are independently hydrogen, C₁₋₆ alkyl or together with the nitrogen atom to which they are attached form a 5- or 6-membered saturated ring optionally containing a further O, S or NR³ group;
or R⁴ and R⁵ together form a 5- or 6-membered saturated ring optionally containing a further O, S or NR³ group and optionally substituted by, C₁₋₆ alkyl;
or pharmaceutically acceptable salts or solvates thereof.

14. The compound according to claim 13, wherein A is a cyclohexane ring.

15. The compound according to claim 13, wherein R¹ and R² together with the nitrogen atom to which they are attached form an unsubstituted morpholine ring or a piperidine ring substituted by a group —(CH₂)ₚ—R⁶ where p and R⁶ are as defined in claim 1.

16. The compound according to claim 13, wherein R³ is hydrogen.

17. The compound according to claim 13, wherein R⁴ is hydrogen.

18. The compound according to claim 13, wherein R⁵ is hydrogen or phenyl optionally substituted by C₁₋₆ alkyl or C₁₋₆ alkoxy.

19. The compound according to claim 13, wherein the compound of formula (I) is selected from:
(1R,2R)—N-[Cyano(2-methoxyphenyl)methyl]-2-(morpholin-4-ylcarbonyl)cyclohexanecarboxamide,
(1R,2R)—N-[Cyano(2-methoxyphenyl)methyl]-2-{[4-(4-fluorobenzyl)piperazin-1-yl]carbonyl}cyclohexane carboxamide,
(1R,2R)—N-[Cyano(2-methoxyphenyl)methyl]-2-(3,4-dihydroisoquinolin-2(1H)-ylcarbonyl)cyclohexane carboxamide,
(±) Trans-N-(cyanomethyl)-2-{[4-(4-fluorobenzyl)piperazin-1-yl]carbonyl}cyclohexanecarboxamide,
(±) Trans-N-[cyano(2-methoxyphenyl)methyl]-2-[(4-methylpiperazin-1-yl)carbonyl]cyclohexanecarboxamide,
(1R,2R)—N-[Cyano(2-methoxyphenyl)methyl]-2-{[4-(4-fluorophenyl)piperazin-1-yl]carbonyl}cyclohexane carboxamide,
(1R,2R)—N-(4-Cyano-1-methylpiperidin-4-yl)-2-{[4-(4-fluorophenyl)piperazin-1-yl]carbonyl}cyclohexane carboxamide,
and pharmaceutically acceptable salts thereof.
